(19) United States
(12) Patent Application Publication
(10) Pub. No.: US 2026/0192087 A1
(43) Pub. Date: Jul. 9, 2026

(54) MEDICAL DEVICE FOR ASPIRATION AND INJECTING FLUID

(71) Applicant: Boston Scientific Scimed, Inc., Maple Grove, MN (US)

(72) Inventor: John Thomas Favreau, Charlton, MA (US)

(73) Assignee: Boston Scientific Scimed, Inc., Maple Grove, MN (US)

(21) Appl. No.: 19/439,010

(22) Filed: Jan. 2, 2026

Related U.S. Application Data
(60) Provisional application No. 63/742,158, filed on Jan. 6, 2025.

(51) Int. Cl. A61M 25/00; A61M 39/24; A61B 1/273

(52) U.S. Cl. CPC A61M 25/0026; A61M 25/0097; A61M 39/24; A61B 1/273; A61M 2025/0037; A61M 2202/0007; A61M 2202/04; A61M 2205/3331

(57) ABSTRACT

Medical devices, medical device systems, and methods are disclosed herein. A medical device, such as a catheter, may include a port, a guidewire port, and a valve assembly including a first one-way valve and a second one-way valve. Within the hub, a wall may define an injection lumen in fluid communication with the first one-way valve and an aspiration lumen in fluid communication with the second one-way valve. A guidewire lumen may extend distally from the guidewire port and may be in fluid communication with the aspiration lumen.

### CROSS-REFERENCE TO RELATED APPLICATIONS

The application claims the benefit of U.S. Provisional Patent Application Serial No. 63/742,158 filed on January 6, 2025, the disclosure of which is incorporated herein by reference.

### TECHNICAL FIELD

The present disclosure is directed to medical devices and associated methods. More particularly, the present disclosure relates to aspiration and injection devices, systems, arrangements and associated methods

### BACKGROUND

Endoscopic retrograde cholangiopancreatography (ERCP) is a technique that utilizes endoscopy and fluoroscopy to diagnose, ascertain, and treat conditions affecting the biliary and/or pancreatic ductal systems and related anatomical structures.

In an ERCP procedure, an endoscope is inserted through a patient or subject’s mouth and advanced down the esophagus. From the esophagus, the endoscope traverses past the stomach through the pylorus (which connects the stomach to the duodenum), into the duodenum, through the ampulla of Vater and the sphincter of Oddi. A desired treatment and/or diagnostic site is then imaged via one or more cameras installed on and/or within the endoscope. A catheter and/or cannula is inserted into the patient or subject and passed through the ampulla of Vater. Contrast solution may be injected into the desired treatment and/or diagnostic site, such as within the bile ducts and/or pancreatic duct.

Cannulas and catheters for use in ERCP procedures may be of single-lumen or multi-lumen design (i.e., containing multiple lumens and/or conduits). There exists a need in the art for improved medical devices, systems and methods for use in ERCP procedures and other procedures.

### BRIEF SUMMARY

This disclosure provides design, material, method, and use alternatives for medical devices, including, but not limited to, medical devices and medical device systems for use in ERCP procedures.

An example medical device may include a hub, the hub including a port and a wall separating an injection lumen of the hub from an aspiration lumen of the hub, a first one-way valve in fluid communication with the port and the injection lumen, and a second one-way valve in fluid communication with the port and the aspiration lumen.

Alternatively or additionally to any of the examples disclosed herein, the first one-way valve may permit fluid flow in a first direction and prohibit fluid flow in a second direction, and the second one-way valve may permit fluid flow in the second direction and prohibit fluid flow in the first direction.

Alternatively or additionally to any of the examples disclosed herein, the first one-way valve may permit fluid flow in a direction that is proximal-to-distal and prohibit fluid flow in a direction that is distal-to-proximal.

Alternatively or additionally to any of the examples disclosed herein, the second one-way valve permits fluid flow in a direction that is distal-to-proximal and prohibits fluid flow in a direction that is proximal-to-distal.

Alternatively or additionally to any of the examples disclosed herein, the medical device may further include an elongate shaft extending in a distal direction from the hub, wherein the wall is configured to couple and/or align with a dividing wall of the elongate shaft.

Alternatively or additionally to any of the examples disclosed herein, the dividing wall may at least partially define a first shaft lumen in fluid communication with the injection lumen and at least partially defines a second shaft lumen in fluid communication with the aspiration lumen.

Alternatively or additionally to any of the examples disclosed herein, the hub may define a guidewire lumen in communication with the second shaft lumen.

Alternatively or additionally to any of the examples disclosed herein, the injection lumen may have a mean cross-sectional area that is less than a mean cross-sectional area of the aspiration lumen.

In an example, a medical device may include a hub, the hub including a first port and a second port, a first valve in fluid communication with the first port, a second valve in fluid communication with the first port, and an elongate shaft extending from the hub, wherein the first valve permits flow in a first direction along a first lumen of the elongate shaft and wherein the second valve permits flow in a second direction along a second lumen of the elongate shaft.

Alternatively or additionally to any of the examples disclosed herein, the hub may include a guidewire lumen extending distally from the second port.

Alternatively or additionally to any of the examples disclosed herein, the medical device may include a seal configured to block fluid through the guidewire lumen.

Alternatively or additionally to any of the examples disclosed herein, the guidewire lumen may be in communication with the second lumen.

Alternatively or additionally to any of the examples disclosed herein, the first valve may prohibit flow in a proximal direction and the second valve may prohibit flow in a distal direction.

Alternatively or additionally to any of the examples disclosed herein, the first lumen may have a mean cross-sectional area that is less than a mean cross-sectional area of the second lumen.

Alternatively or additionally to any of the examples disclosed herein, the first lumen may be configured to receive injection fluid and the second lumen may be configured to receive a negative pressure and a guidewire.

In an example, a medical device may include a hub, the hub including a first port, a second port, a valve assembly in fluid communication with the first port and comprising a first one-way valve and a second one-way valve, a wall defining a first lumen in fluid communication with the first one-way valve and a second lumen in fluid communication with the second one-way valve, and a third lumen extending distally from the second port and in communication with the third lumen.

Alternatively or additionally to any of the examples disclosed herein, the first one-way valve may permit fluid flow in a proximal-to-distal direction and prohibit fluid flow in a distal-to-proximal direction and the second one-way valve may permit fluid flow in the distal-to-proximal direction and prohibit fluid flow in the proximal-to-distal direction.

Alternatively or additionally to any of the examples disclosed herein, the first lumen may have a mean cross-sectional area that is less than a mean cross-sectional area of the second lumen.

Alternatively or additionally to any of the examples disclosed herein, the medical device may further include an elongate shaft extending in a distal direction from the hub, wherein the elongate shaft may include a first shaft lumen and a second shaft lumen, the first shaft lumen may be configured to align with the first lumen and the second shaft lumen may be configured to align with the second lumen and the third lumen.

Alternatively or additionally to any of the examples disclosed herein, the second shaft lumen may be configured to simultaneously receive a vacuum pressure and a guidewire.

This summary is an overview of some of the teachings of the present application and is not intended to be an exclusive or exhaustive treatment of the present subject matter. Further details are found in the detailed description and appended claims. Other aspects will be apparent upon reading and understanding the following detailed description and viewing the drawings that form a part thereof, each of which is not to be taken in a limiting sense.

### BRIEF DESCRIPTION OF THE SEVERAL VIEWS OF THE DRAWINGS

The drawings illustrate the design and utility of configurations of the present disclosure. It should be noted that the figures are not drawn to scale and that elements of similar structures or functions are represented by like reference numerals throughout the figures. The drawings are not to be considered as limiting the scope of the disclosure. The present disclosure is described and explained with additional specificity and detail through the use of the accompanying drawings, in which:

FIG. 1 illustrates a schematic view of an example medical device;

FIG. 2 illustrates a schematic cross-sectional view of a portion of a hub, taken along line 2-2 in FIG. 1;

FIG. 3 illustrates a schematic cross-sectional view of the portion of the hub depicted in FIG. 2, taken along line 3-3;

FIG. 4 illustrates a schematic cross-sectional view of a portion of the medical device depicted in FIG. 1;

FIG. 5 illustrates a schematic cross-sectional view of an example elongate shaft of a medical device; and

FIG. 6 illustrates a schematic diagram of an example method of using an illustrative medical device.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g., 1 to5 includes 1,1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “a configuration”, “some configurations”, “other configurations”, etc., indicate that the configuration described may include one or more particular features, structures, and/or characteristics. However, such recitations do not necessarily mean that all configurations include the particular features, structures, and/or characteristics. Additionally, when particular features, structures, and/or characteristics are described in connection with one configuration, it should be understood that such features, structures, and/or characteristics may also be used in connection with other configurations whether or not explicitly described unless clearly stated to the contrary.

The following detailed description should be read with reference to the drawings in which similar elements in different drawings are numbered the same. The drawings, which are not necessarily to scale, depict illustrative configurations and are not intended to limit the scope of the disclosure.

Cannulas and catheters (hereinafter “catheters”) for use in ERCP procedures may be of single-lumen or multi-lumen design (e.g., containing multiple lumens and/or conduits) and include one or more lumens for aspiration, fluid injection, and traversing a guidewire. However, each of single-lumen catheters and multi-lumen catheters have flaws. For example, although a single-lumen design may have a simple design and decrease forces needed to aspirate or inject fluid, air bubbles may be unintentionally introduced into a subject during procedures due to combined paths for a guidewire and fluid injection, which may cause harm to the subject and/or may obstruct the view of a practitioner through a scope during the procedure.

Multi-lumen catheters may have separate lumens running through the body of the catheter (e.g., along the catheter length) which separate the injection fluid, aspiration fluid, and the guidewire. The multiple lumens offer greater injection performance due to the ability to prime injection channels and lack of opportunity for the guidewire to pull air into the injection ports and injection channels and/or lumens. However, multi-lumen catheter designs may require that an axial cross-section of the aspiration lumen be very small to make room for the guidewire lumen, therefore increasing a force needed to aspirate through the catheter, which may hinder procedures using a multi-lumen catheter.

Given the deficiencies of single-lumen and multi-lumen (i.e., having multiple lumens) catheters, and the potential hazards posed to a patient or subject in either implementation, it has been found that a catheter configured as disclosed herein satisfies many needs in the art. Disclosed herein are devices, systems and methods relating to a medical device that facilitates achieving a desired amount, velocity and pressure of injection fluid (or other like medium) to a target site while also facilitating providing a desired amount, velocity, and negative pressure (i.e., suction) of aspiration from the target site. Other advantages of the present disclosure will be described further herein.

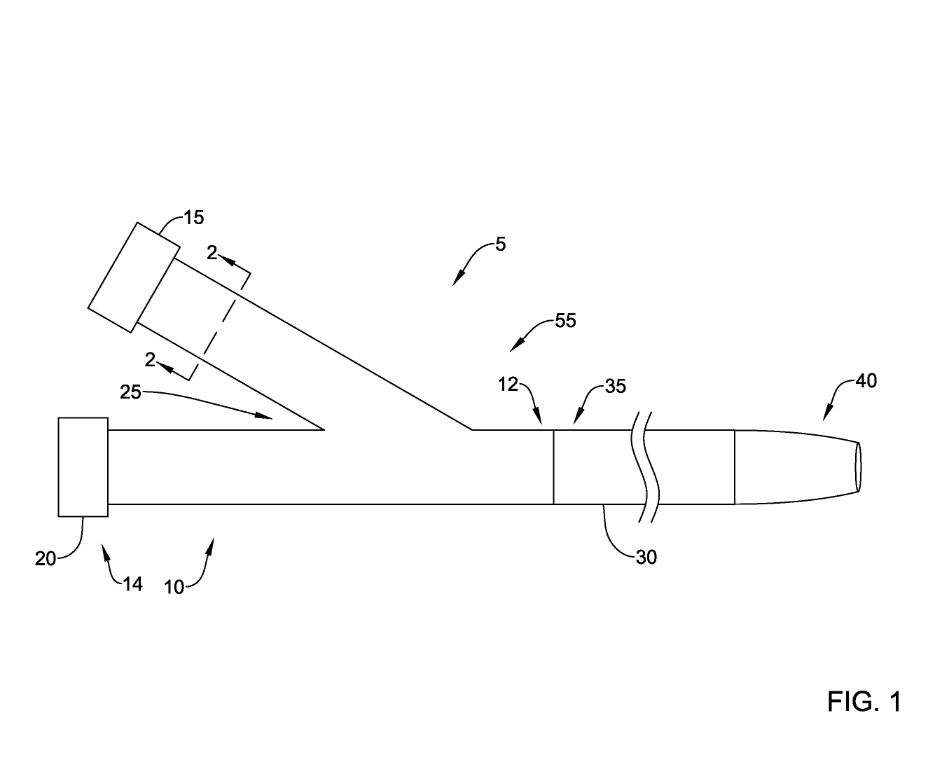

FIG. 1 depicts an example medical device (e.g., a catheter 5) in accordance with at least one configuration. The catheter 5 may include a hub 10 with one or more ports and an elongate shaft 30 extending in a distal direction from the hub 10. In some examples, the hub 10 may include a bifurcation 25, a first port 15 (e.g., a fluid port or other suitable port), and a second port 20 (e.g., a guidewire port or other suitable port), among other suitable components. The first port 15 and the second port 20 may be branches of the hub 10 and/or proximal ends of branches of the hub 10 at which a component may couple with or interact with the hub 10. Although FIG. 1 depicts the bifurcation 25 distal of proximal portions of both of the first port 15 and the second port 20, the bifurcation 25 may be configured in one or more other suitable manners along the hub 10. In some examples, the bifurcation 25 may be present farther distally or farther proximally in relation to one or more other components of the hub 10.

The elongate shaft 30 may be coupled with the hub 10 in any suitable manner. In some examples, the elongate shaft 30 may be connected to, coupled with, adhered to, anchored to, and/or otherwise secured relative to the hub 10. This connection, coupling, adherence, anchoring, and/or bond may occur at a distal end 12 of the hub 10 and/or at points proximal of the distal end 12 of the hub 10.

The elongate shaft 30 may include one or more shaft lumens, and the one or more shaft lumens may extend through and/or along the elongate shaft 30 from a proximal end 35 of the elongate shaft 30 to a distal end 40 of the elongate shaft 30, or may discontinue at any point therethrough. The one or more shaft lumens may be disposed in a coaxial relationship, a concentric relationship, a parallel relationship, a side-by-side relationship, an intertwined relationship, or in any other suitable relationship. In some examples, the elongate shaft 30 may be an elongate tube defining one or more shaft lumens, but other suitable configurations of the elongate shaft 30 and/or the one or more shaft lumens are contemplated.

The first port may be located at or extend to a proximal end 14 of the hub 10. In some examples, the first port 15 of the hub 10 may be configured to couple with or otherwise be in fluid communication with one or more fluid sources configured to initiate a fluid flow through the hub 10. In some examples, the fluid source may be configured to provide fluid to a lumen of the hub 10 and/or elongate shaft 30 and/or initiate an aspiration flow through a lumen of the hub 10 and/or the elongate shaft 30. In some examples, the first port 15 may be configured to couple with one or more fluid sources including, but not limited to, a pump device, a suction device, an aspiration pump, a syringe, a syringe pump, and/or other suitable device for initiating fluid flow in the lumens of the hub 10 and/or the elongate shaft 30.

The first port 15 may be configured to couple with the fluid source in any suitable manner. For example, the first port 15 may be configured to couple with the fluid source using one or more couplings including, but not limited to, a luer connection, a threaded connection, a bayonet connection, and/or other suitable type of connection.

The second port 20 may be located at or extend to a proximal end 14 of the hub 10, which may be a guidewire port and may accept passage of a guidewire or other like device through one or more guidewire lumens in communication with the second port 20. In some examples, the second port 20 at the hub 10 may be configured to receive a guidewire in an over-the-wire manner. Alternatively or additionally, the second port 20 may be located distal of the first port 15 and/or of the hub 10 or an additional port may be located distally of the hub 10 such that the catheter 5 may engage a guidewire through a side port of the elongate shaft 30 in a rapid exchange configuration.

A guidewire lumen extending from the second port 20 in the hub 10 (or other port) may be in communication with a lumen in communication with the first port 15 at any point within hub 10 or distal of the hub 10. In some examples, the guidewire lumen extending from the second port 20 may merge with or otherwise first communicate with the lumen extending from within the first port 15 at a point distal of the bifurcation 25. Other suitable configurations of the lumens extending from the first port 15 and the second port 20 are contemplated.

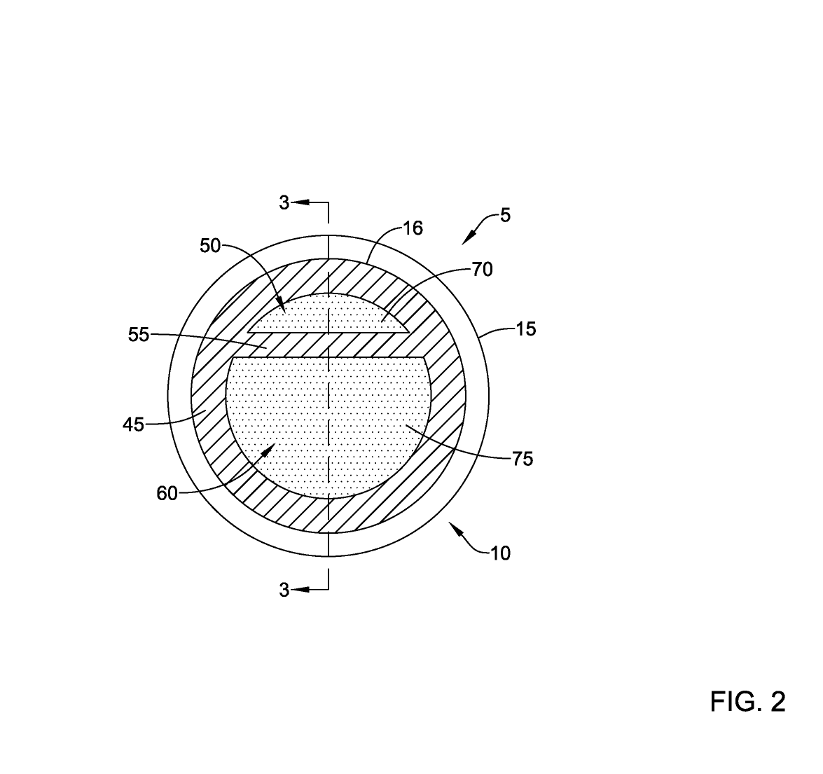

FIG. 2 depicts a cross-section of a portion of the hub 10, including the first port 15, taken along line 2-2 in FIG. 1. An outer wall 45 of the hub 10 may entirely or at least partially define one or more lumens extending through the hub 10. In some examples, the outer wall 45 may be or may form at least part of a shell or housing 16 (hereinafter housing 16) of the hub 10.

The hub 10 (e.g., the first port 15 or other portion of the hub 10) may include a wall 55 (e.g., an inner or radial wall) separating an injection lumen 50 (e.g., a first lumen) from an aspiration lumen 60 (e.g., a second lumen). The wall 55 may be connected to, coupled to, adhered to, anchored to, or otherwise bonded to the outer wall 45 at any portion of the outer wall 45 and by any suitable technique in the art. In some examples, the wall 55 may be made integral with or may be formed monolithically with the outer wall 45. In one example, the wall 55 and the outer wall 45 may be part of a one-piece construction of the housing 16 of the hub 10, as depicted for example in FIG. 2.

The outer wall 45 may have any suitable thickness. In some examples, a thickness of the outer wall 45 may be in a range of 0.005 inches (0.127 millimeters (mm)) to 0.009 inches (2.286 mm), although thicknesses outside of this range are contemplated for wall 45.

The housing 16 of the hub 10 may be formed from any suitable material. In some examples, the housing 16 may be formed from one or more of a rigid material, a flexible material, a polymer material, a metal material, and/or other suitable types of material. In one example, the housing 16 may be formed, at least in part, from a rigid polymer material.

The injection lumen 50 may be provided for the transfer and passage of injection fluid, and/or other fluids or like mediums designed to travel through the elongate shaft 30 and be delivered out of the distal end 40 of the elongate shaft 30 to a target site of or in the subject. In some examples, the fluids may be injected through the injection lumen 50 at high velocities or pressures. A size of a cross-sectional area of the injection lumen 50 may be configured to maintain desired velocities or pressures of the fluid injected into the injection lumen 50 as the fluid travels through the catheter 5. As shown in FIG. 2, the cross-sectional area of the injection lumen 50 may be smaller than the cross-sectional area of the aspiration lumen 60, but other suitable configurations are contemplated. In this and other examples, the cross-sectional area of the injection lumen 50 may be in a range of about 0.0008 in² (0.516128 mm²) to about 0.0022 in² (1.419352 mm²). In additional and/or alternative non-limiting examples, the cross-sectional area of the injection lumen 50 at any point along the injection lumen 50 may be less than 0.0008 in² (0.516128 mm²) or may be greater than 0.0022 in² (1.419352 mm²). In some examples, the injection lumen 50 and the aspiration lumen 60 may have a continuous, sustained, and/or substantially continuous, and/or substantially sustained geometry, such that a mean cross-sectional area (i.e., the average of the areas of any number of cross-sections and/or cross-sectional slices) of the injection lumen 50 may be smaller than a mean cross-sectional area of the aspiration lumen 60, but other suitable configurations are contemplated.

The injection lumen 50 may have any suitable length. In some examples, the injection lumen 50 may extend from the first port 15 to the distal end 40 of the elongate shaft 30, but the injection lumen 50 may extend other suitable distances.

The injection lumen 50, in addition to the other lumens disclosed and/or referred to herein, may be configured and/or otherwise adapted to be connected to a fluid source including, but not limited to, a pump, a syringe, a syringe pump, and/or other suitable fluid source. In some examples, the fluid sources may be in communication (i.e., fluid communication) with the injection lumen 50 at the first port 15 using a direct connection or indirect connection (e.g., intermediate tubing, etc.)

The aspiration lumen 60 may be provided for the transfer and passage of fluid from the target site of or within the subject at the distal end 40 of the elongate shaft 30 through the elongate shaft 30 and the first port 15. In some examples, the solids and/or fluids may be aspirated through the aspiration lumen 60. A size of a cross-sectional area of the aspiration lumen 60 may be configured to maintain desired velocities or pressures within the aspiration lumen 60 to aspirate desired fluid (e.g., flowing liquid, gases, and/or solids) from the target site and out of the first port 15. In this and other examples, the cross-sectional area of the aspiration lumen 60 may be in a range of range from about 0.0007787 in² (0.5024 mm²) to about 0.0304 in² (19.625 mm²). In one example, the cross-sectional area of the aspiration lumen 60 may be in a range of range from about 0.004867 in² (3.14 mm²) to about 0.01095 in² (7.065 mm²). In other non-limiting examples, the cross-sectional area of the aspiration lumen 60 at any point along the aspiration lumen 60 may be less than 0.0007787 in² (0.5024 mm²) and may be greater than 0.0304 in² (19.625 mm²).

The aspiration lumen 60 may have any suitable length. In some examples, the aspiration lumen 60 may extend from the first port 15 to the distal end 40 of the elongate shaft 30, but the aspiration lumen 60 may extend other suitable distances.

The aspiration lumen 60, in addition to the other lumens disclosed and/or referred to herein, may be configured and/or otherwise adapted to be connected to a fluid source capable of establishing a vacuum pressure including, but not limited to, suction devices and/or pump devices such as pumps, aspiration pumps, vacuum pumps, syringes, syringe pumps and/or other suitable fluid source. In some examples, the fluid sources may be in communication with the aspiration lumen 60 at the first port 15 using a direct connection or indirect connection (e.g., intermediate tubing, intermediate conduits, intermediate couplings, etc.) The same fluid source coupled with the first port 15 may be used to inject fluid through the injection lumen 50 and aspirate fluid through the aspiration lumen 60 or two or more fluid sources may be separately coupled with the first port 15 to inject fluid through the injection lumen 50 and aspirate fluid through the aspiration lumen 60.

One or more valves may form a valve assembly and may be located in or otherwise configured to control fluid flow through the injection lumen 50 and/or the aspiration lumen 60. In some examples, the valves in or controlling fluid flow through the injection lumen 50 and/or the aspiration lumen 60 may be one-way valves and/or other suitable type of valve.

The one or more valves or valve assemblies may be located at any suitable location in the catheter 5 that is in communication with the first port 15 and the injection lumen 50 or other suitable injection lumen and the aspiration lumen 60 or other suitable aspiration lumen. In some examples, a first valve 70 may be in fluid communication with the first port 15 and the injection lumen 50 and a second valve 75 may be in fluid communication with the first port and the aspiration lumen 60. In some examples, the one or more valves may be located in the hub 10 at a location in or distal of the first port 15, as depicted in FIG. 2 showing a cross-sectional view of the first valve 70 and the second valve 75. In one example, a first valve 70 may be located in the injection lumen 50 and a second valve 75 may be located in the aspiration lumen 60. In another example, the first valve 70 may be located in the first port 15 proximal of and in fluid communication with the injection lumen 50 and the second valve 75 may be located in the first port 15 proximal of and in fluid communication with the aspiration lumen 60. In some examples, the first valve 70 and/or the second valve 75 may be located in the elongate shaft 30 or other location distal of the hub 10 and in fluid communication an injection lumen and/or aspiration lumen of the elongate shaft. The first valve 70 and the second valve 75 may be located at a same axial location of the catheter 5 or at different axial locations of the catheter. Other suitable positioning of the valves of the valve assembly in the catheter 5 are contemplated.

When the first valve 70 is configured as a one-way valve in or proximate the injection lumen 50, the first valve 70 may permit fluid flow (i.e., flow) in a first direction (e.g., in a proximal-to-distal direction, such as from the practitioner to deeper within the patient and/or away from the practitioner’s end of the catheter 5) and prohibit fluid flow in a second direction (e.g., in a distal-to-proximal direction, such as from deeper within the patient and/or toward the practitioner’s end of the catheter 5). When the second valve 75 is configured as a one-way valve in or proximate the aspiration lumen 60, the second valve 75 may permit fluid flow in the second direction (e.g., in the distal-to-proximal direction), and prohibit fluid flow in the first direction (e.g., the proximal-to-distal direction). Other suitable configurations of the valves are contemplated.

Although certain examples have been described that include single one-way valves in one or more lumens described herein, other combinations of valves and lumens are contemplated. In some examples, each of the injection lumen 50 and the aspiration lumen 60 may have one or more valves that are not one-way valves. In some examples, each of the injection lumen 50 and the aspiration lumen 60 may have two or more one-way valves. In yet other examples, the injection lumen 50 may have one or more one-way valves, while the aspiration lumen 60 may have only one one-way valve. In other examples, the injection lumen 50 may have only one one-way valve and the aspiration lumen 60 may have one or more one-way valves. In some examples, the one or more valves in communication with the injection lumen 50 and/or the aspiration lumen 60 may be located proximal of a proximal end of the injection lumen 50 and/or the aspiration lumen 60. Other suitable examples of the lumens and valves of the catheter 5 are contemplated.

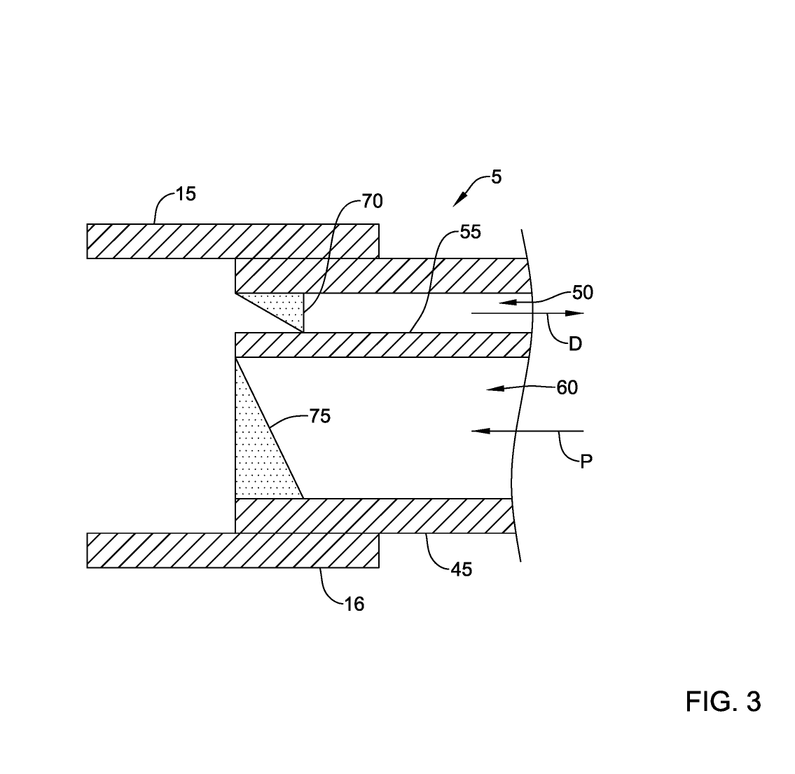

FIG. 3 depicts a cross-section of the hub 10 taken along line 3-3 in FIG. 2. The first valve 70 may be included within injection lumen 50. The second valve 75 may be included within the aspiration lumen 60. In some examples, additional and/or alternative valves may be included, and may be incorporated anywhere within the first port 15, the second port 20, the injection lumen 50, the aspiration lumen 60, the guidewire lumen, the elongate shaft 30, and/or at one or more other suitable locations along the catheter 5.

The first valve 70 and/or the second valve 75 may be any suitable type of valve. Example suitable types of valves include, but are not limited to, one-way valves, a check valve, a duckbill valve, an umbrella valve, a pneumatic valve, a ball check valve, a stopcock, a mechanical valve, an electrical valve, an elastic valve, a polymeric valve, a metallic valve, an actuated valve, a gravity valve, a pressure valve, a rotating valve, a ball check valve, a swing check valve, a fluidic valve, a spring check valve, a fluidic valve, and/or other suitable type of valve. In some examples and as discussed, the first valve 70 positioned in or otherwise at a location in communication with the injection lumen 50 may be a one-way valve configured to permit fluid flow in a proximal-to-distal (i.e., upstream) direction D while prohibiting fluid flow in a distal-to-proximal (i.e., downstream) direction P within injection lumen 50. In some examples, the second valve 75 positioned in or otherwise at a location in communication with the aspiration lumen 60 may be a one-way valve configured to permit fluid flow in the distal-to-proximal direction P while prohibiting fluid flow in the proximal-to-distal direction D within the aspiration lumen 60.

The wall 55 may divide and/or separate the injection lumen 50 from the aspiration lumen 60, as depicted for example in FIG. 3. In some examples, the wall 55 may at least partially define one or more of the injection lumen 50 and aspiration lumen 60. The wall 55 may have any suitable thickness, including but not limited to a thickness ranging from 0.005 inches (0.127 mm) to 0.009 inches (0.2286 mm), although thicknesses outside of this range are also contemplated for wall 55.

The wall 55 may have any suitable configuration. In some examples, the wall 55 may be linear, straight, or may have one or more curves, convexities, concavities, and/or may vary in thickness. In one example, the outer wall 45 may support the wall 55 (e.g., as depicted in FIG. 2) and/or may accept a bond and/or coupling with the wall 55. In one example, two or more walls 55 may be present and may define the injection lumen 50, the aspiration lumen 60, and/or the guidewire lumen of the catheter 5.

As discussed, the first valve 70 and/or the second valve 75 may be located at any suitable location along the hub 10 or other portion of the catheter 5. In some examples, the first valve 70 and the second valve 75 may be located at the same longitudinal location along the hub 10, as depicted for example in FIG. 3. In one example, the first valve 70 may be positioned farther distally and/or farther proximally in relation to the hub 10 and hub components described herein than the second valve 75. Alternatively, the first the valve 70 and/or the second valve 75 may be positioned farther distally and/or farther proximally in relation to the other one of the first valve 70 and/or the second valve 75. In some example configurations, the first valve and/or the second valve 75 may be positioned farther distally and/or farther proximally in relation to the first port 15 and/or the injection lumen 50 or the aspiration lumen 60. In one example, the first valve 70 and the second valve 75 may positioned within an insert located in the first port 15 and aligned with the injection lumen 50 and the aspiration lumen 60, respectively. In some examples, the first valve 70 and the second valve 75 may be or may be part of a single valve assembly or valve component. Other suitable configurations of the valves of the catheter 5 are contemplated.

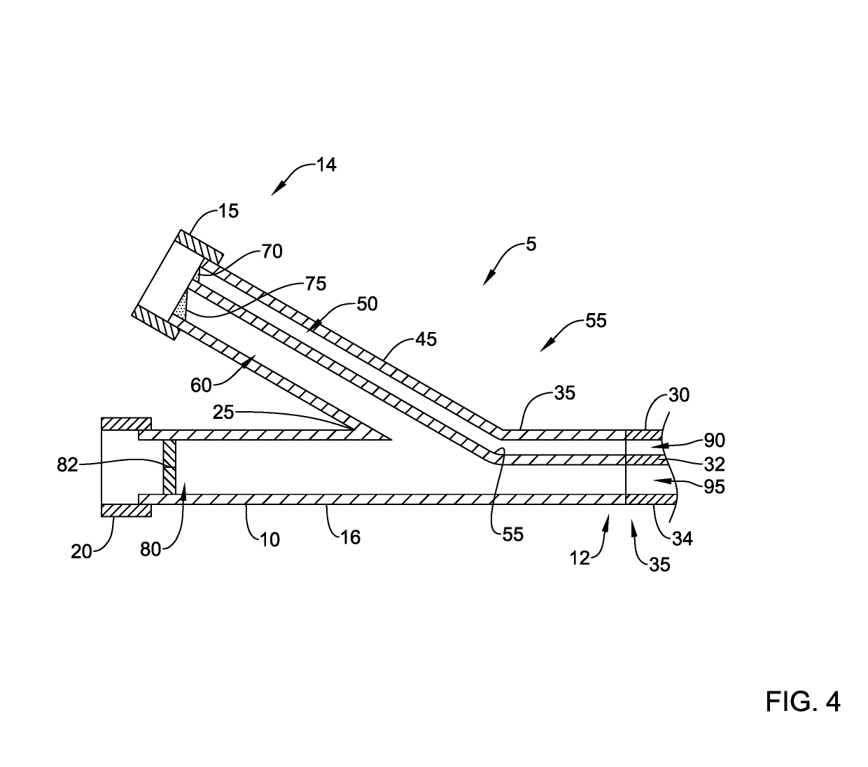

FIG. 4 depicts a schematic cross-section of a proximal portion of an illustrative configuration of the catheter 5. In this and other examples, the catheter 5 may include the hub 10 which includes the first port 15, the second port 20, and the bifurcation 25. In some examples and as depicted for example in FIG. 4, the first port 15 and/or the second port 20 may have a different configuration than (e.g., may be formed from a different material than) a different or more distal portion of the respective port 15, 20 or the hub 10 to facilitate engaging or receiving a component or accessory device and/or for other suitable purposes. At or distal of the first port 15, the first valve 70 may be disposed at or within the injection lumen 50 and the second valve 75 may be disposed at or within the aspiration lumen 60. The wall 55 may separate the injection lumen 50 from the aspiration lumen 60, as discussed.

The second port 20 may include or otherwise be in communication with a guidewire lumen 80 (e.g., a third lumen) extending in a distal direction along or from the second port 20. The guidewire lumen 80 may extend within the second port 20 and may originate at the same point as the second port 20 or at some point or collection of points distal of and/or proximate to the second port 20. The guidewire lumen 80 may have a cross-sectional area at along its length that is configured to accommodate a guidewire having a diameter in a range of 0.025 inches (0.635 mm) to 0.035 inches (0.889 mm), although the guidewire lumen 80 may be configured to accommodate guidewires having a diameter outside of the range.

The guidewire lumen 80 may include one or more seals 82 for blocking fluid flow and/or impeding fluid flow through the second port 20 or for one or more other purposes (e.g., wiping the guidewire, etc.) The one or more seals 82 may be positioned anywhere within guidewire lumen 80 and/or proximate to guidewire lumen 80 and may be one-way valves, two-way valves, three-way valves, four-way valves, gaskets, rings, plugs, wipers, slit seals, elastomeric seals, polymeric seals, or other seals known in the art. The one or more seals 82 within the guidewire lumen 80 may be located anywhere within guidewire lumen 80 and may be separated by any distance and/or pattern of distances. The one or more seals 82 may be omitted.

As discussed, the elongate shaft 30 may be coupled with the hub 10. The elongate shaft 30 may be coupled with the hub 10 in any suitable manner including, but not limited to, a butt-joint coupling, an over mold coupling, a weld coupling, a reflow coupling, an adhesive coupling, and/or one or more other suitable coupling.

The elongate shaft 30 may have any suitable outer diameter (OD), which may vary or remain constant along the length of the elongate shaft 30. In some examples, the OD of the elongate shaft 30 may be in a range of about 0.09 inches (2.286 mm) to about 0.165 inches (4.191 mm), in a range from about 0.09 inches (2.286 mm) to about 0.110 inches (2.794 mm), and/or may be within one or more other suitable ranges extending outside of these ranges.

The elongate shaft 30 may include one or more lumens extending an entire length of the elongate shaft 30 or along portions thereof. In some examples, the elongate shaft 30 may include a first shaft lumen 90 (e.g., an injection lumen, etc.) extending from the proximal end 35 of the elongate shaft 30 to the distal end 40 (not shown in FIG. 4) of the elongate shaft 30 and a second shaft lumen 95 (e.g., an aspiration lumen, a guidewire lumen, etc.) extending from the proximal end 35 of the elongate shaft 30 to the distal end 40 of the elongate shaft 30.

The first shaft lumen 90 and the second shaft lumen 95 may have any suitable configuration relative to one another. For example, the first shaft lumen 90 or the second shaft lumen 95 may be concentric with, coaxial with, side-by-side with, or run parallel with the other one of the second shaft lumen 95 and the first shaft lumen 90. In one example and as depicted in FIG. 4, the first shaft lumen 90 and the second shaft lumen 95 may extend side-by-side with one another and parallel to one another, but other suitable configurations are contemplated. In some examples, the first shaft lumen 90 may reside superior (i.e., above) the second shaft lumen 95 or vice-versa, and may carry and transport injection fluid or the like therein from the injection lumen 50 of the hub 10. In some examples, the second shaft lumen 95 may reside inferior (i.e., below) the first shaft lumen 90 or vice-versa, and may carry and transport aspiration fluid, aspiration materials, or other like fluids, suspensions, or materials therein to the aspiration lumen 60 of the hub 10. In some examples, the first shaft lumen 90 may be offset at an angle from second shaft lumen 95. In some examples, the first shaft lumen 90 may be centrally located or located near and/or proximate outer wall 105. In some examples, the second shaft lumen 95 may be centrally located, or located near and/or proximate outer wall 105. In some examples, the second shaft lumen 95 may occupy 50-95% of the interior of the elongate shaft 30 to facilitate aspirating fluid or other suitable material from a target site and/or receive a guidewire, while the first shaft lumen 90 may occupy 5-50% of the interior of the elongate shaft 30 to facilitate injection fluid to a target site at or proximate the distal end 40 of the elongate shaft 30, or vice-versa. In some examples, first shaft lumen 90 may occupy 10-90% of the interior of the elongate shaft 30, and second shaft lumen 95 may occupy 10-90% of the interior of the elongate shaft 30. In these instances, percentage of the interior refers to the percentage of interior cross-sectional area occupied.

The first shaft lumen 90 may have any suitable shape or configuration. In some examples, the first shaft lumen 90 may be round, circular, oval, egg-shaped, arc-shaped, concave, convex, arcuate, triangular, diamond-shaped, inverted-diamond-shaped, square, polygonal, and may maintain these cross-sectional profiles over the entire length, perimeter or one or more portions of the length or perimeter of the first shaft lumen 90.

The second shaft lumen 95 may have any suitable shape or configuration. In some examples, the second shaft lumen 95 may be round, circular, oval, arc-shaped, mouth-shaped, concave, convex, arcuate, triangular, diamond-shaped, inverted-diamond-shaped, square, polygonal, and may maintain these cross-sectional profiles over the entire length, perimeter, or one or more portions of the length or perimeter of the second shaft lumen 95. In some examples, these characteristics may apply to additional or alternative lumens of the catheter 5.

One or more dividing walls 32 and/or outer walls 34 of the elongate shaft 30 may define the first shaft lumen 90 and/or the second shaft lumen 95. The dividing wall 32 and/or the outer wall 34 along the elongate shaft 30 may have any suitable configurations. In some examples, the outer wall 34 and the one or more dividing walls 32 of the elongate shaft 30 may have consistent thicknesses along a length of the elongate shaft 30 or may have variable thicknesses along the length of the elongate shaft 30. In some examples, the dividing wall(s) 32 and the outer walls 34 may be configured to establish a desired first cross-sectional area of the first shaft lumen 90 of the elongate shaft 30 and a desired second cross-sectional area of the second shaft lumen 95. For example, the dividing wall 32 and the outer wall 34 of the elongate shaft 30 may be configured such that the first shaft lumen 90 may have a smaller area, a smaller cross-sectional area, a smaller mean cross-sectional area, and/or contain a smaller volume than a cross-sectional area, a mean cross-sectional area, and/or a volume of the second shaft lumen 95. In some examples, a third lumen may be provided in the elongate shaft 30. In some examples, four or more lumens may be provided and may each have varying areas, cross-sectional areas, mean cross-sectional areas, and/or contain differing volumes from one another. In some examples, some, none, one or more, or all of the lumens may have varying areas, varying cross-sectional areas, varying mean cross-sectional areas, and/or contain differing volumes from one another.

When the hub 10 is coupled with the elongate shaft 30, as schematically depicted for example in FIG. 4, the wall 55 and/or the outer wall 45 of the hub 10 may be aligned with a dividing wall 32 and/or exterior walls (e.g., an outer wall 34 of the elongate shaft 30) of or defining the first shaft lumen 90 and second shaft lumen 95 of the elongate shaft 30. Additional dividing walls and/or lumens are contemplated and configured and/or otherwise adapted to align and/or couple with the wall 55 and outer wall 45 of the hub 10, and/or align with other suitable features of the hub 10.

When the hub 10 is coupled with the elongate shaft 30, the aspiration lumen 60 may combine with the guidewire lumen 80 and be combined with or be in fluid communication with the second shaft lumen 95 of the elongate shaft 30. In some examples, the aspiration lumen 60 and the guidewire lumen 80 of the hub 10 may combine to form a single lumen at a location distal of the bifurcation 25, as depicted for example in FIG. 4, or at one or more other suitable locations. In some examples, the aspiration lumen 60 and the guidewire lumen 80 may merge multiple times (e.g., two or more times), and may do so along the length of the hub 10 and/or elongate shaft 30 (e.g., as part of the second shaft lumen 95). In one example, a guidewire or other like device may enter the catheter 5 through the second port 20, pass through the guidewire lumen 80, and pass into, within, and/or through the combined aspiration and guidewire lumen 60, 80, and into the second shaft lumen 95 of the elongate shaft 30. Fluid and/or other material may be aspirated from a target site through the second shaft lumen 95 and/or the aspiration lumen 60 while the guidewire is extending through the second shaft lumen 95 and/or the guidewire lumen 80 and/or with the guidewire removed from the second shaft lumen 95 of the elongate shaft 30 and/or the guidewire lumen 80.

The elongate shaft 30 may be made from any material or combination of materials known in the art and feasible for the purposes of the disclosure herein. For example, the elongate shaft 30 may be formed from materials including, but not limited to, polymers, metals, flexible materials, malleable materials, durable materials, heat-resistant materials, cold-resistant materials, ductile materials, rigid materials, semi-conductive materials, conductive materials, insulative materials, dielectric materials, materials or compositions with a combination of these aforementioned criteria may be applied, and/or other suitable materials.

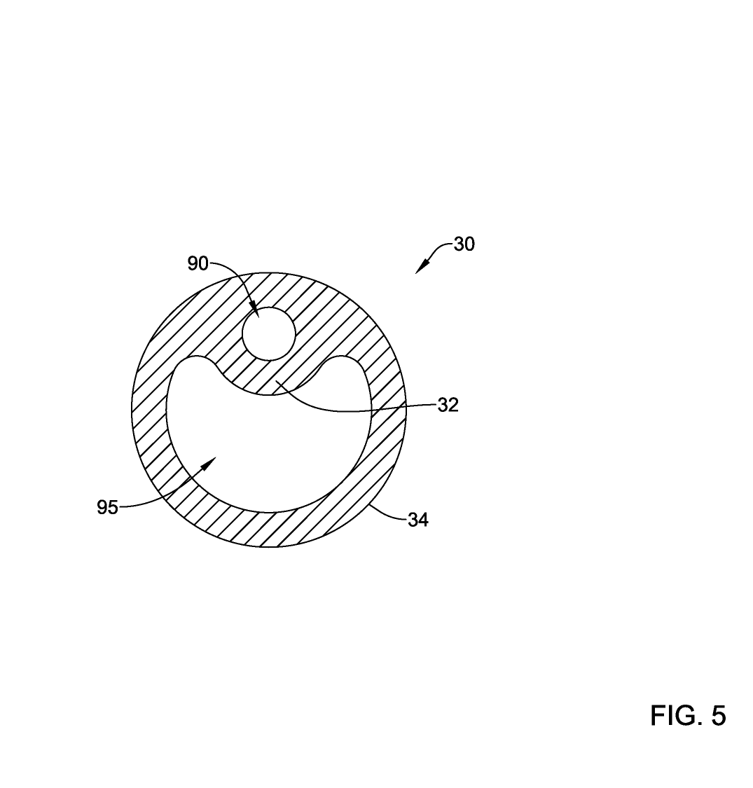

FIG. 5 depicts a schematic cross-section view of an illustrative configuration of the elongate shaft 30 having the first shaft lumen 90 and the second shaft lumen 95. As depicted in FIG. 5, the first shaft lumen 90 and the second shaft lumen 95 may be separated from one another by the dividing wall 32 and at least partially defined by the outer wall 34. In the example configuration depicted in FIG. 5, the first shaft lumen 90 may be configured to deliver an injection fluid to a target site at or proximate the distal end 40 of the elongate shaft 30 and have a smaller cross-sectional area (e.g., a smaller mean cross-sectional area) than a cross-sectional area (e.g., a mean cross-sectional area) of the second shaft lumen 95, which may be configured to receive a negative pressure therein, aspiration material from the target site, and a guidewire extending proximal through the guidewire lumen 80 and the second port 20. Such a configured elongate shaft 30 may facilitate delivering injection fluid to a target site while mitigating a risk of creating air bubbles, while providing a lumen of sufficient size for receiving a guidewire aspirated material from the target site. Other suitable configurations of the first shaft lumen 90 and the second shaft lumen 95 of the elongate shaft 30 are contemplated.

The first shaft lumen 90 may have any suitable cross-sectional area. In some examples, the first shaft lumen 90 may have the same, similar, or different cross-sectional area than the cross-sectional area of the injection lumen 50 of the hub 10. In this and other examples, the cross-sectional area of the first shaft lumen 90 may be in a range of about 0.0008 in² (0.516128 mm²) to about 0.0022 in² (1.419352 mm²). In additional and/or alternative non-limiting examples, the cross-sectional area of the first shaft lumen 90 at any point along the first shaft lumen 90 may be less than 0.0008 in² (0.516128 mm²) or may be greater than 0.0022 in² (1.419352 mm²).

The second shaft lumen 95 may have any suitable cross-sectional area. In some examples, the second shaft lumen 95 may have the same, similar, or different cross-sectional area than the cross-sectional area of the aspiration lumen 60 of the hub 10. In some examples, the second shaft lumen 95 may be configured to accommodate aspiration from a target site while a guidewire extends therethrough. In this and other examples, the cross-sectional area of the second shaft lumen 95 may be in a range of range from about 0.0007787 in² (0.5024 mm²) to about 0.0304 in² (19.625 mm ²). In one example, the cross-sectional area of the second shaft lumen 95 may be in a range of range from about 0.004867 in² (3.14 mm²) to about 0.01095 in² (7.065 mm²). In other non-limiting examples, the cross-sectional area of the second shaft lumen 95 at any point along the second shaft lumen 95 may be less than 0.0007787 in² (0.5024 mm²) and may be greater than 0.0304 in² (19.625 mm²).

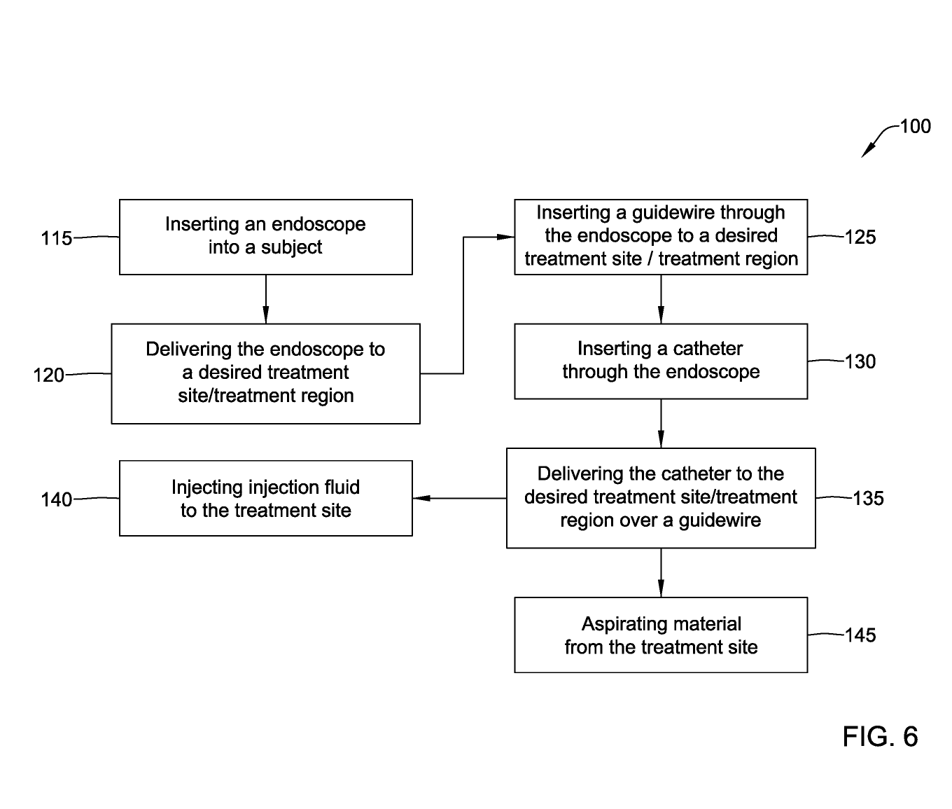

FIG. 6 displays a schematic diagram of an illustrative method 100 of using a catheter with an endoscope. Other suitable methods of using the catheter disclosed herein are contemplated.

In the method 100, an endoscope may be inserted 115 into a subject (e.g., orally inserted into the subject) and delivered 120 to a desired target site (e.g., treatment site) and/or desired treatment region of a subject or patient. After, before or during positioning the endoscope at a desired location of the treatment region, a guidewire may be inserted 125 into the endoscope, through the endoscope, and into the desired treatment region. A catheter may be inserted 130 into the endoscope, over the guidewire when the guidewire is utilized, and into the patient or subject. In some examples, the catheter may be delivered 135 to the target site and/or desired treatment region over the guidewire, which may extend beyond a distal end of the endoscope. In some examples, the inserting 125 of the guidewire may be omitted or one or more other suitable devices may be utilized to guide the catheter to the treatment site and/or the treatment region.

The catheter may be any suitable catheter. In some examples, the catheter may include one or more features described herein including, but not limited to, an elongate shaft having a two lumens, a hub with a first port and a second port, an injection lumen and an aspiration lumen extending in fluid communication with the first port, a one-way valve controlling flow through the injection lumen and allowing fluid flow in the distal direction and blocking fluid flow in the proximal direction, a one-way valve controlling flow through the aspiration lumen and allowing fluid flow in the proximal direction and blocking fluid flow in the distal direction, a guide wire lumen that combines with the aspiration lumen, and/or other suitable features.

Once the catheter is positioned with a distal end at the target site, injection fluid may be injected 140 to or otherwise provided to the target site through the injection lumen of the catheter. In some examples, a fluid source containing the injection fluid may be coupled to the first port at a proximal end of the catheter and in fluid communication with the injection lumen. In response to actuation of the fluid source, the injection fluid may pass the one-way valve in fluid communication with the injection lumen within the hub of the catheter and pass through the inflation lumen to the target site. The injection fluid provided by the fluid source at the first port may be blocked from passing through the aspiration lumen by the one-way valve configured to prevent distal flow of fluid through the aspiration lumen.

Once the catheter is positioned with the distal end at the target site, fluid may be aspirated 145 from the treatment site through the aspiration lumen of the catheter. In some examples, a fluid source configured to aspirate fluid from the target site (e.g., configured to create a negative pressure in the aspiration lumen) may be coupled to the first port at the proximal end of the catheter and may be in fluid communication with the aspiration lumen. In response to actuation of the fluid source, a negative pressure may be created at the first port and an aspiration fluid flow may be created through the one-way valve in fluid communication with the aspiration lumen within the hub of the catheter to aspirate material from the target site through the aspiration lumen to the fluid source or suitable receptacle, while the one-way valve in communication with the injection lumen prevents an aspiration flow or negative pressure in the injection lumen due to preventing flow in the proximal direction.

Aspirating 145 with the catheter may be performed before, during, or after injecting 145 fluid to target site. In some examples including for ERCP procedures and/or other procedures, aspirating 145 material from the treatment site may occur prior to injecting 140 fluid to the target site. For example, when performing an ERCP procedure, aspirating 145 material from the treatment site may occur prior to injecting fluid to the treatment site to confirm whether the catheter is at the target site (e.g., is in the bile duct) based on the type of material aspirated (e.g., if the material aspirated is bile, it is confirmed the catheter or cannula is in the bile duct). Then, once the catheter has been confirmed to be at the target site, fluid may be injected to the target site.

Aspirating 145 with the catheter may occur while the guidewire is extending through a lumen in fluid communication with the aspiration lumen of the hub of the catheter, while the guidewire is being removed from the lumen in fluid communication with the aspiration lumen, and/or after the guidewire has been removed from the lumen in fluid communication with the aspiration lumen. A combined guidewire and aspiration lumen of the catheter may be sized to accommodate aspirating material from the target site while the guidewire is extending through the combined guidewire and aspiration lumen.

Methods as discussed above and described herein may have their steps performed sequentially, concurrently, or in any combination or permutation of the aforementioned. Other suitable features or steps of the methods discussed herein are contemplated.

In an example of using the catheter 5, the catheter 5 as described herein may be implemented in procedures such as endoscopic retrograde cholangiopancreatography (ERCP) and other like procedures and/or procedures involving the anatomical structures and systems described herein. An endoscope may be inserted into a subject or patient and delivered to a desired treatment site and/or region such as a biliary duct. The endoscope may image the biliary duct subsequently to or concomitantly with the delivery of the catheter 5. The catheter 5 may be delivered to the desired treatment site and/or region and/or may be delivered proximate to, proximally of, or distally of the desired treatment site and/or region through the endoscope and/or over a guidewire.

In an example of using the catheter 5, the distal end 40 of the elongate shaft 30 may be placed proximate to, proximally of, or distally of the desired treatment site and/or region within a subject. Injection fluid may be delivered through port 15 and further through injection lumen 50 by passing through the first valve 70 and along the first shaft lumen 90 of the elongate shaft 30 before arriving at the desired treatment site and/or region and/or after arriving at the desired treatment site. Subsequently or concurrently, aspiration fluid and/or aspirate or other like material may be removed from the treatment site via the second shaft lumen 95 and through the aspiration lumen 60 by passing through the second valve 75 in the port 15.

In an example, a guidewire may be placed within, passed through, or reside within the second port 20 and may further advance into the guidewire lumen 80, the aspiration lumen 60, and the second shaft lumen 95 if so desired. Additionally, a guidewire may be retracted within, retracted through, or retractably reside within second shaft lumen 95, aspiration lumen 60, the guidewire lumen 80, and/or the second port 20.

In an example, a negative pressure (i.e., vacuum, vacuum pressure) source may be connected to aspiration lumen 60 to aid in aspirating fluid and other like materials through the second shaft lumen 95 of the elongate shaft 30, back through aspiration lumen 60, out of port 15 and out of hub 10. Any known pumps and/or vacuum sources may be incorporated for aspiration or other like techniques.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The materials that can be used for the various components of the systems presently disclosed may include those commonly associated with medical devices. For simplicity purposes, the following discussion makes reference to the catheter 5 referenced above. However, this is not intended to limit the devices and methods described herein, as the discussion may be applied to other similar devices and/or components of devices disclosed herein.

The catheter 5 and/or components thereof may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium- molybdenum alloys (e.g., UNS: R30035 such as MP35-N® and the like), nickel- molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

Some examples of suitable polymers may include, but are not limited to, polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some configurations the sheath can be blended with a liquid crystal polymer (LCP). For example, the mixture can contain up to about 6 percent LCP.

In some configurations, a degree of Magnetic Resonance Imaging (MRI) compatibility is imparted into catheter 5. For example, the catheter 5 or portions thereof may be made of a material that does not substantially distort the image and create substantial artifacts (i.e., gaps in the image). Certain ferromagnetic materials, for example, may not be suitable because they may create artifacts in an MRI image. The catheter 5 or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

Tubular and/or elongate components of the catheter 5 may include one or more tubular members that may have slots formed therein. Various configurations of arrangements and configurations of slots are contemplated. For example, in some configurations, at least some, if not all of the slots are disposed at the same or a similar angle with respect to the longitudinal axis of the tubular and/or elongate components of the catheter 5. The slots can be disposed at an angle that is perpendicular, or substantially perpendicular, and/or can be characterized as being disposed in a plane that is normal to the longitudinal axis of the tubular and/or elongate components of the catheter 5. However, in other configurations, the slots can be disposed at an angle that is not perpendicular, and/or can be characterized as being disposed in a plane that is not normal to the longitudinal axis of the tubular components of the catheter 5. Additionally, a group of one or more the slots may be disposed at different angles relative to another group of one or more the slots. The distribution and/or configuration of the slots can also include, to the extent applicable, any of those disclosed in U.S. Pat. No. US 7,914,467, the entire disclosure of which is herein incorporated by reference. Some example configurations of appropriate micromachining methods and other cutting methods, and structures for tubular members including slots and medical devices including tubular members are disclosed in U.S. Pat. Publication Nos. 2003/0069522 and 2004/0181174-A2; and U.S. Pat. Nos. 6,766,720; and 6,579,246, the entire disclosures of which are herein incorporated by reference. Some example configurations of etching processes are described in U.S. Pat. No. 5,106,455, the entire disclosure of which is herein incorporated by reference. It should be noted that the methods for manufacturing the catheter 5 may include forming the slots in the tubular or elongate components of the catheter 5 using these or other manufacturing steps.

The present disclosure has been described above based on various configurations. Various modifications can be made to the combination of the components and processes in the configurations and that such modifications are included in the scope of the present disclosure.

While configurations of the present disclosure have been described above, it is to be understood that variations and modifications will be apparent to those skilled in the art without departing from the scope and spirit of the disclosure. The scope of the present disclosure, therefore, is to be determined solely by the following claims.

What is claimed is:

1. A medical device, comprising:
a hub, the hub including a port and a wall separating an injection lumen of the hub from an aspiration lumen of the hub;
a first one-way valve in fluid communication with the port and the injection lumen; and
a second one-way valve in fluid communication with the port and the aspiration lumen.

2. The medical device of claim 1, wherein the first one-way valve permits fluid flow in a first direction and prohibits fluid flow in a second direction, and the second one-way valve permits fluid flow in the second direction and prohibits fluid flow in the first direction.

3. The medical device of claim 1, wherein the first one-way valve permits fluid flow in a direction that is proximal-to-distal and prohibits fluid flow in a direction that is distal-to-proximal.

4. The medical device of claim 1, wherein the second one-way valve permits fluid flow in a direction that is distal-to-proximal and prohibits fluid flow in a direction that is proximal-to-distal.

5. The medical device of claim 1, further comprising:
an elongate shaft extending in a distal direction from the hub, and
wherein the wall is configured to couple and/or align with a dividing wall of the elongate shaft.

6. The medical device of claim 5, wherein the dividing wall at least partially defines a first shaft lumen in fluid communication with the injection lumen and at least partially defines a second shaft lumen in fluid communication with the aspiration lumen.

7. The medical device of claim 6, wherein the hub defines a guidewire lumen in communication with the second shaft lumen.

8. The medical device of claim 1, wherein the injection lumen has a mean cross-sectional area that is less than a mean cross-sectional area of the aspiration lumen.

9. A medical device, comprising:
a hub, the hub including a first port and a second port;
a first valve in fluid communication with the first port;
a second valve in fluid communication with the first port; and
an elongate shaft extending from the hub;
wherein the first valve permits flow in a first direction along a first lumen of the elongate shaft and wherein the second valve permits flow in a second direction along a second lumen of the elongate shaft.

10. The medical device of claim 9, wherein the hub includes a guidewire lumen extending distally from the second port.

11. The medical device of claim 10, further comprising a seal configured to block fluid through the guidewire lumen.

12. The medical device of claim 10, wherein the guidewire lumen is in communication with the second lumen.

13. The medical device of claim 9, wherein the first valve prohibits flow in a proximal direction and the second valve prohibits flow in a distal direction.

14. The medical device of claim 9, wherein the first lumen has a mean cross-sectional area that is less than a mean cross-sectional area of the second lumen.

15. The medical device of claim 9, wherein the first lumen is configured to receive injection fluid, and the second lumen is configured to receive a negative pressure and a guidewire.

16. A medical device, comprising:
a hub, the hub including:
a first port;
a second port;
a valve assembly comprising a first one-way valve and a second one-way valve;
a wall defining a first lumen in fluid communication with the first one-way valve and a second lumen in fluid communication with the second one-way valve; and
a third lumen extending distally from the second port and in communication with the second lumen.

17. The medical device of claim 16, wherein:
the first one-way valve permits fluid flow in a proximal-to-distal direction and prohibits fluid flow in a distal-to-proximal direction; and
wherein the second one-way valve permits fluid flow in the distal-to-proximal direction and prohibits fluid flow in the proximal-to-distal direction.

18. The medical device of claim 16, wherein the first lumen has a mean cross-sectional area that is less than a mean cross-sectional area of the second lumen.

19. The medical device of claim 16, further comprising:
an elongate shaft extending in a distal direction from the hub, and
wherein the elongate shaft includes a first shaft lumen and a second shaft lumen, the first shaft lumen is configured to align with the first lumen and the second shaft lumen is configured to align with the second lumen and the third lumen.

20. The medical device of claim 19, wherein the second shaft lumen is configured to simultaneously receive a vacuum pressure and a guidewire.
